(19) United States
(12) Patent Application Publication
(10) Pub. No.: US 2004/0014034 A1
(43) Pub. Date: Jan. 22, 2004

(54) Method of producing a recombinant virus

(76) Inventors: David H. Evans, Edmonton (CA); Xiao-Dan Yao, Guelph (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/455,695

(22) Filed: Jun. 6, 2003

Related U.S. Application Data
(60) Provisional application No. 60/385,886, filed on Jun. 6, 2002.

(51) Int. Cl. C12Q 1/70; C12P 19/34; C12N 7/02; C12N 15/86

(52) U.S. Cl. 435/5; 435/91.2; 435/239; 435/456

(57) ABSTRACT

The present invention relates to methods and kits for modifying viral genomes. The method involves introducing into a host cell containing a helper virus, two or more fragments of a first viral genome, the fragments having ends that are capable of being joined together comprising as little as basepair of overlapping sequence. The helper virus is able to facilitate recombination and reactivation of the DNA fragments into active infectious virions.

This application claims the benefit under 35 USC §119(e) from U.S. Provisional patent application serial No. 60/385,886, filed Jun. 6, 2002.

### FIELD OF THE INVENTION

The present invention relates to the methods of producing genetically modified viruses which replicate in the cytoplasm of a host cell.

### BACKGROUND OF THE INVENTION

Poxviruses are very large DNA viruses that replicate in the cytoplasm of infected cells. Because of interest in the poxvirus variola as the causative agent of smallpox, poxvirus research has a long history dating back the beginnings of modern virology. Some of the earliest experiments described a process called “non-genetic reactivation” wherein cells infected by one poxvirus can promote the recovery of a second virus rendered non-infectious on its own by heat, ultraviolet light or other treatment (8, 15). A characteristic feature of this reaction is that the two viruses need not be genetically identical, for example vaccinia virus will reactivate variola virus and myxoma virus will reactivate rabbit fibroma virus. Although the process of non-genetic reactivation has never been characterized in molecular detail, it is generally assumed that the helper virus provides the enzymatic machinery necessary to uncoat, transcribe, repair, and perhaps replicate the inactivated virus, complementing in trans other virion components inactivated by heat or other treatments.

Subsequent experiments have shown that replicating poxviruses can also reactivate poxviruses from transfected virus DNA and several applications of the process have been described which facilitate the production of recombinant viruses. Sam and Dumbell originally demonstrated that one orthopoxvirus could be used to reactivate the DNA of a second virus in a “homologous” packaging reaction (15). Scheiflinger et al. subsequently showed that cells infected with fowlpox virus could reactivate transfected vaccinia virus DNA in a “heterologous” packaging scheme and exploited the narrow host range of fowlpox virus to simplify the rescue and packaging of vaccinia recombinants prepared in vitro by DNA ligation (16). Although the method is elegant and has been used in other studies (2, 9, 10), this approach produced recombinant chimeras at efficiencies of only 6-14% and the added technical complexities associated with propagating fowlpox virus have seemingly limited its widespread adoption. A recent publication suggests ways in which the efficiency can be enhanced substantially through the use of a psoralen-inactivated helper virus (19), although this homologous packaging reaction risks recombination between two vaccinia virus genomes of which one has been subjected to highly mutagenic pre-treatment.

In most of these studies, some care seems to have been taken to extract and restrict virus DNA in ways that minimize shearing the 190-kbp-vaccinia genome. Yet, no matter how carefully this is done, it is difficult to imagine poxvirus DNA surviving the transfection process intact and thus the reactivation process presumably repairs transfected viral DNA using the recombination systems readily detected in poxvirus-infected cells. This raises questions concerning the role of recombination in poxvirus reactivation reactions. There remains a need for a method of modifying a viral genome in a simple and efficient manner.

### SUMMARY OF THE INVENTION

The present inventors have shown that replicating poxviruses can exploit a single strand annealing reaction to produce simple recombinants from mixtures of co-transfected virus and PCR-amplified DNAs, as well as complex recombinants from multiple overlapping fragments of virus DNA. These observations show that heterologous reactivation reactions can be used to genetically manipulate the structure of poxvirus genomes in ways not previously appreciated. It also suggests a secure way in which existing collections of infectious virus stocks could be replaced by archives consisting of stable and biologically harmless overlapping clones.

Accordingly, the present invention provides a method of producing a first recombinant virus comprising:

(a) providing a host cell that is infected with a second virus;

(b) introducing two or more nucleic acid fragments from the first virus into the host cell, wherein said two or more nucleic acid fragments have ends that are capable of being joined;

(c) incubating the host cell under conditions to allow the nucleic acid fragments to recombine and form a recombinant virus; and

(d) recovering the recombinant virus.

In embodiments of the present invention, each of the two or more nucleic acid fragments comprises between 10-9000 basepair (bp), preferably between 14-100 bp, more preferably between 16-20 bp, of sequence that is homologous to the fragment to which it is to be joined.

Also provided are kits for performing the method of the invention.

The present invention provides a new method for modifying the genome of a virus that does not involve the use of traditional DNA ligation techniques, nor the preparation of recombinant plasmids.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic showing an embodiment of the method of the invention for rescuing recombinant vaccinia virus using cells infected with a helper Shope fibroma virus. Examples of such cell lines include BGMK and SIRC. A mix of SFV and vaccinia are recovered from the infected cell but these are easily separated by plating on cell lines that support only the growth of vaccinia virus. One such line is BSC-40.

FIG. 2 is a schematic showing the reconstruction of an intact virus genome from an overlapping array of subgenomic DNA fragments, PCR fragments, or randomly sheared molecules. The “X's” show sites of recombination.

FIG. 3 is a schematic showing an embodiment of the method of the present invention for rearranging poxvirus genomes by the selective use of modified PCR fragments and overlap recombination. The “patch” fragment shares homology with the adjacent fragments, but introduces a precisely determined deletion which is indicated in brackets.

FIG. 4 is a schematic showing an example of a method for creating a patch fragment. The extra “tails” on primers b and c are complementary to each other. This method deleted all of the DNA lying between primers b and c. The two PCR products share end homology (“X”) and can be fused into a single recombinant DNA in a second PCR. An application of this method for making deletion viruses is further illustrated in FIG. 13.

FIG. 5 shows reactivation of transfected vaccinia DNA in SIRC cells infected with SFV. Reactivated virus were plated on BSC-40 cells to select for growth of vaccinia virus. The vaccinia virus genome bore a gpt-selectable marker not encoded by the reactivating SFV. The presence of the gpt marker was demonstrated by plating with or without selection.

FIG. 6 shows a Southern blot analysis of vaccinia virus genomes reactivated using a heterologous SFV helper virus. DNA was extracted from 6 different reactivated viruses, digested with HindIII, size-fractionated by electrophoresis, and Southern blotted using randomly-labelled XY-I-SceIVV DNA as a probe. The HindIII fragment pattern, characteristic of the vaccinia WR parent strain (lane 7), were reproduced in all of the reactivated viruses.

FIG. 7 shows a pulsed field gel analysis of untreated and restricted vaccinia virus DNAs. . DNA was extracted from vaccinia virions, size fractionated using pulsed field agarose gel electrophoresis, and stained with ethidium bromide.

FIG. 8 is a schematic illustration of the vaccinia virus genome. Panel A shows restriction sites (vaccinia Copenhagen); Panel B shows potential PCR amplicons; Panel C shows potential integration sites in the NotI- or I-Scel-modified TK locus.

FIG. 9 is a schematic illustrating the replacement of portions of the vaccinia genome with PCR-amplified DNA's. The BgII B fragment is replaced by a 15.1 kbp PCR amplicon. Another fragment serves to repair a second BgII-induced double-strand break.

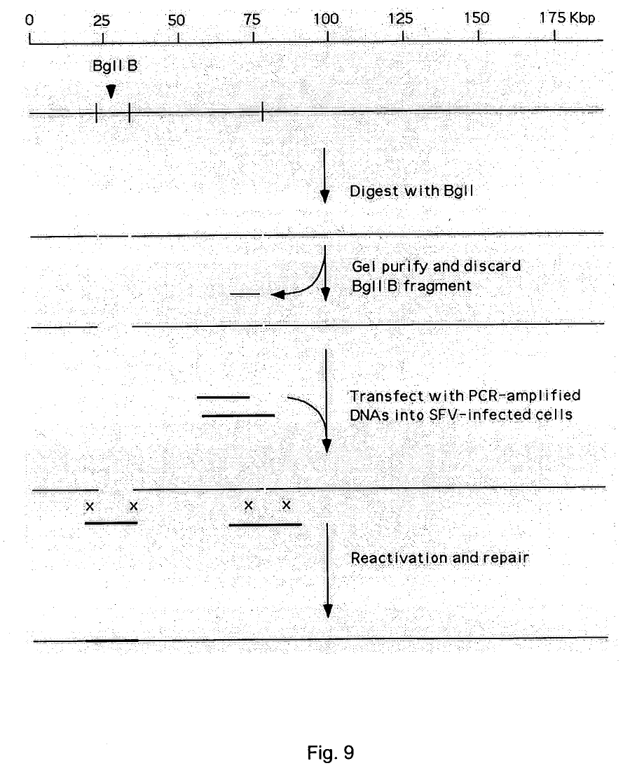

FIG. 10 illustrates double-stranded break repair in SFV-infected cells. The method permits targeting a DNA fragment encoding a lacZ cassette into a double stranded break created by digesting a modified vaccinia virus genome with I-Scel. The Southern blot shown in the lower panel illustrates the resulting LacZ+ virus were all genetic recombinants.

FIG. 11 shows the effects of DNA concentration and homology length on the efficiency of recombinant virus production. Increasing the length of homology to 50 bp on either end of the targeting fragment can generate 100% recombinant virus under optimal conditions.

FIG. 12 shows the single-step construction of recombinant vaccinia viruses expressing green fluorescent protein. The yield of recombinant virus is sufficiently high that recombinants can be detected directly without further plaque purification. The upper panel shows the targeting strategy, the lower panel illustrates the recombinant virus produce active green fluorescent protein in the presence of a T7 RNA polymerase encoding helper virus.

FIG. 13 shows how the method can be used to construct a vaccinia deletion virus.

FIG. 14 shows the combinations of DNAs that were tested to examine reactivation of vaccinia virus from co-transfected mixtures of PCR amplified DNAs and vaccinia restriction fragments. A PCR fragment (4L), encoding essential vaccinia genes, generated as many reactivated and recombinant virus as did a natural DNA fragment encoding the same genes (Pmel-B).

### DETAILED DESCRIPTION OF THE INVENTION

Method of the Invention

The present inventors have devised a way in which cells infected by one “helper” virus can be used to reactivate a second virus introduced into infected cells as DNA fragments. The capacity to reconstruct a live virus from an assemblage of natural and PCR-amplified DNA fragments provides a novel way in which one can genetically manipulate the structure of poxvirus in dramatic ways not previously considered possibly.

The present inventors have shown that cells infected with Shope fibroma virus (SFV) catalyze very high efficiency recombination reactions that require surprisingly little homology between recombining linear molecules. The present inventors have further demonstrated that these SFV-infected cells can reactivate transfected vaccinia virus DNA and produce simple recombinants of the kinds described previously. These results have led to the development of a new method of modifying and constructing recombinant viruses.

Accordingly, the present invention provides a method of producing a first recombinant virus comprising:

(a) providing a host cell that is infected with a second virus;

(b) introducing two or more nucleic acid fragments from the first virus into the host cell, wherein said two or more nucleic acid fragments have ends that are capable of being joined;

(c) incubating the host cell under conditions to allow the nucleic acid fragments to recombine and form a recombinant virus; and

(d) recovering the recombinant virus.

The phrase “two or more nucleic acid fragments from the first virus” means that the nucleic acid molecules are derived or obtained from a viral genome. The nucleic acid fragments may be obtained from DNA extracted from the virus using standard techniques. The extracted DNA can be digested with restriction enzymes to prepare the nucleic acid fragments. The nucleic acid fragments can also be amplified using the polymerase chain reaction (PCR). The nucleic acid fragments are preferably at least 50 bp in length and generally from about 50 bp to 50,000 bp in length, more preferably from about 500 bp to 20,000 bp in length.

The phrase “wherein said two or more nucleic acid fragments have ends that are capable of being joined” means that the fragments will have overlapping regions of homology that will allow them to be recombined or joined under the appropriate conditions. Preferably, the region of homology will be between 10-9000 basepair (bp), preferably between 12-100 bp, more preferably between 16-20 bp.

The first virus is preferably from the family Poxviridae which includes the subfamilies Chordopoxvirinae and Entomopoxvirinae. The Poxvirdae is preferably a Chordopoxvirnae which includes the genuses: avipoxvirus (which includes species canarypox virus; fowlpox virus; Hawaiian goose poxvirus; pigeonpox virus; and vultur gryphus poxvirus); capripoxvirus (which includes species capripoxvirus strain Ranipet; goatpox virus; lumpy skin disease virus; and sheeppox virus); leporipoxvirus (which includes species malignant rabbit fibroma virus; myxoma virus; rabbit fibroma virus and Shope fibroma virus); molluscipoxvirus (which includes species molluscum contagiosum virus); orthopoxvirus (which includes species aracatuba virus; BeAn 58058 virus; Buffalopox virus; camelpox virus; cantagalo orthopoxvirus; cowpox virus; ectromelia virus; elephantpox virus; monkeypox virus; rabbitpox virus; raccoonpox virus; skunkpox virus; taterapox virus; vaccinia virus; variola virus (smallpox virus); and volepox virus); parapoxvirus (which includes species bovine popular stomatitis virus; orf virus; pseudocowpox virus; red deer parapoxvirus; and sealpox virus); suipoxvirus (which includes species swinepox virus) and yatapoxvirus (which includes species tanapox virus; yaba monkey tumor virus; and yaba-like disease virus). Most preferably the first virus is selected from the genus orthopoxvirus or leporipoxvirus. In a specific embodiment, the first virus is selected from the genus orthopoxvirus, more specifically the species vaccinia virus.

The second virus may be from any virus that can catalyze trans-acting replication, recombination, and virus reactivation reactions of the first virus. The second virus is preferably from the family Poxviridae as described above for the first virus. Most preferably, the second virus is selected from the genus leporipoxvirus or orthopoxvirus. In a specific embodiment, the second virus is selected from the genus leporipoxvirus, more specifically the species Shope fibroma virus (SPV). The second virus may also include known inactivated helper viruses, such as for example, heat, UV-light, or psoralen-inactivated vaccinia virus.

The first and second viruses are preferably not from the same species, most preferably not from the same genus of poxvirus. In one embodiment, the first virus is from the genus orthopoxvirus and the second virus is from the genus leporipoxivirus. In another embodiment, the first virus is from the genus leporipoxivirus and the second virus is from the genus orthopoxvirus.

The host cell may be any cell which supports the replication of the first and second viruses. For example, when the first virus is vaccinia virus and the second virus is the Shope fibroma virus (SFV), the host cell may be rabbit or monkey cells, preferably Buffalo african green monkey kidney (BGMK) cells.

The recombinant virus may be recovered using any known technique. In an embodiment of the present invention, the recombinant virus is isolated by plating the host cells, or an extract therefrom, on a cell line that does not support the replication of the second virus. For example, when the first virus is vaccinia, the host cells, or an extract therefrom, may be plated on BSC-40 (African green monkey kidney) or HeLa cells, which only supports the growth of vaccinia. The titers of the virus recovered by the present method are preferably greater than 10² PFU/μg, more preferably, 10⁴ PFU/μg, most preferably greater than 10⁶ PFU/μg.

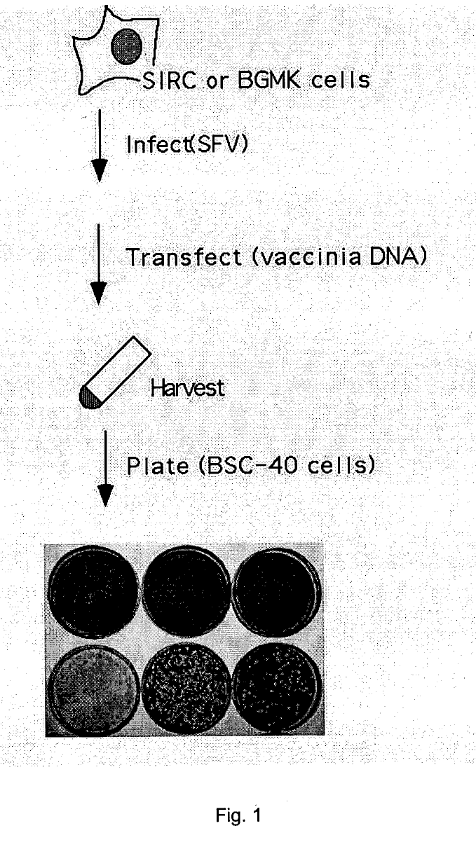

The method of the invention, in its simplest form, is illustrated schematically in FIG. 1.

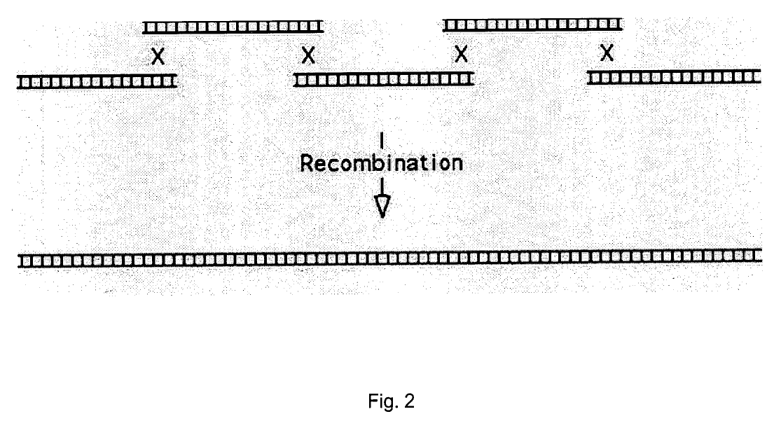

A feature of the method of the present invention is the high recombination frequency. For example, vaccinia DNA can be sheared randomly or cut into different overlapping fragments and the SFV-infected cells are capable of stitching the fragments back together. FIG. 2 illustrates this reaction feature.

Due to the efficiency of this reaction, it has been possible to reconstruct a virus from a whole series of different overlapping fragments. A mixture of overlapping PCR-amplified fragments plus restriction fragments was used to accomplish this task.

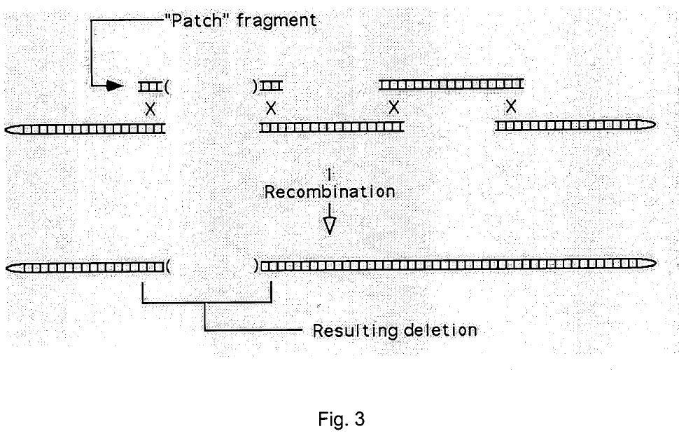

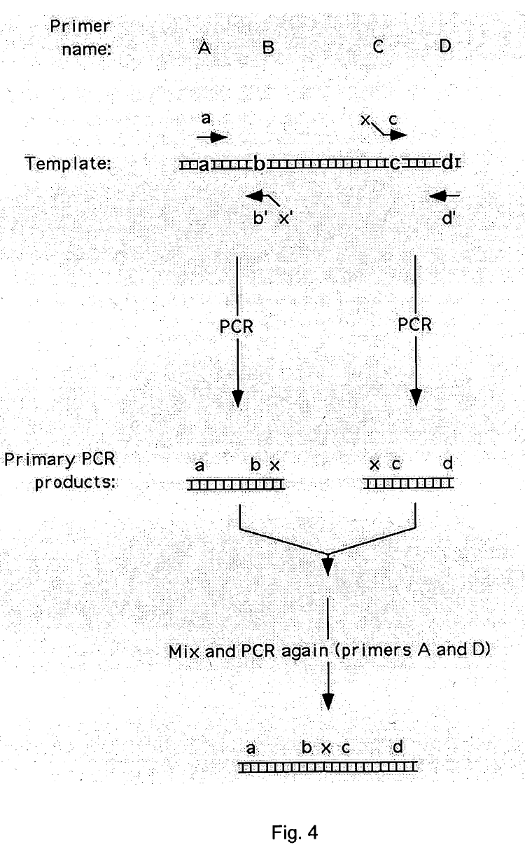

The particular advantage of the method of the present invention is that if viruses can be put back together (“rescued”) from a series of overlapping PCR or restriction fragments, this opens up a whole realm of new routes by which one could rearrange the structure of virus genomes. In particular, interest in viruses as vaccine vectors has been tempered by the presence of undesirable “pathogenes”. Pathogenes are virus genes that are typically not essential for growth in culture, but serve to increase the infectivity of a virus by inhibiting the activities of the immune system. By using a judicious choice of PCR primers and carefully designing overlaps, one can selectively delete many or even all such non-essential and potentially dangerous genes from any given virus. FIG. 3 illustrates the principle of this method. FIG. 4 shows one of several ways of creating “patch” fragments which could be used to introduce deletions into an assemblage of genome fragments. This approach is a useful way of producing “gutted” vectors that should be much safer than traditional virus vaccine vectors. Furthermore, deleting such non-essential genes creates additional space for introducing large transgenes by the same routes. This is of special interest where it is desirous to introduce many different transgenes or antigens into a single virus in a simple and controlled route.

Accordingly, in an embodiment of the present invention there is provided a method of preparing a first recombinant virus having a deletion in a non-essential region comprising:

(a) providing a host cell that is infected with a second virus;

(b) introducing two or more nucleic acid fragments from the first virus into the host cell, wherein said two or more nucleic acid fragments have ends that are capable of being joined, wherein said fragments do not comprise a non-essential region of the virus;

(c) incubating the host cell under conditions to allow the nucleic acid fragments to recombine and form a recombinant virus having a deletion in a non-essential region; and

(d) recovering the recombinant virus.

In a further embodiment of the present invention, the method of the invention can be used. to prepare a recombinant virus containing a heterologous DNA encoding a foreign gene of interest. Accordingly, the present invention further provides a method of producing a first recombinant virus comprising a heterologous nucleic acid sequence encoding a foreign gene of interest comprising:

(a) providing a host cell that is infected with a second virus;

(b) introducing into the host cell (i) two or more nucleic acid fragments from the first virus, wherein said two or more nucleic acid fragments have ends that are capable of being joined and (ii) a heterologous nucleic acid sequence encoding a foreign gene of interest;

(c) incubating the host cell under conditions to allow the nucleic acid fragments to recombine and form a recombinant virus comprising the heterologous nucleic acid sequence; and

(d) recovering the recombinant virus.

The phrase “heterologous nucleic sequence encoding a foreign gene of interest” as used herein may include a DNA sequence that is naturally-occurring in a genome of a eukaryotic cytoplasmic DNA virus, as well as a sequence that is not naturally-occurring in such a genome. Furthermore, a heterologous DNA sequence encoding a foreign gene of interest may comprise only sequences that are naturally-occurring in a eukaryotic cytoplasmic DNA virus, where such a sequence is inserted into a location in the genome of that cytoplasmic DNA virus different from the location where that sequence naturally occurs.

Inserting a heterologous DNA sequence encoding a foreign gene of interest into a eukaryotic cytoplasmic DNA virus genome according to the present invention is useful for the purpose of expressing a desired protein, particularly a human protein. The foreign proteins may be produced in cell cultures, for preparing purified proteins, or directly in human or animal hosts, for immunizing the host with a vaccine comprising a modified virus according to the present invention.

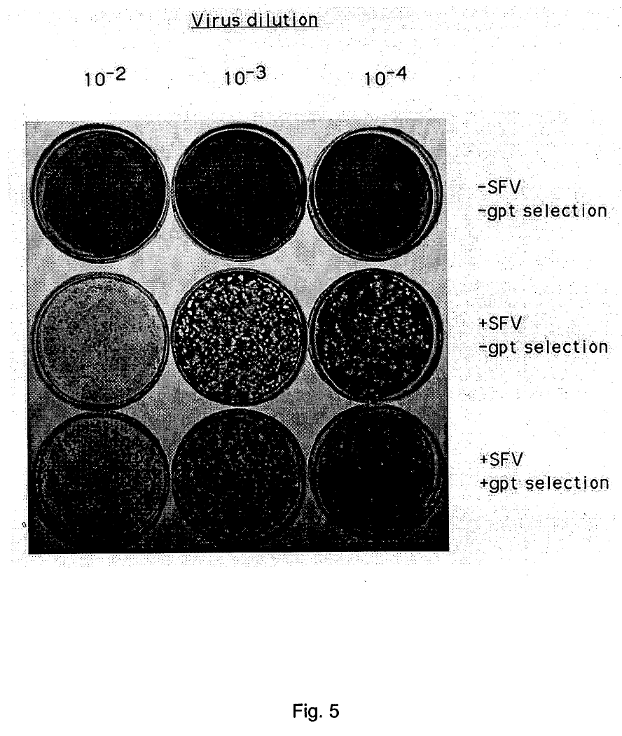

In certain embodiments, the step of modifying a virus genome by inserting a heterologous DNA sequence encoding a foreign gene of interest comprises introducing a marker gene function for distinguishing the recombinant virus from the intact first virus. In one such embodiment, a DNA sequence inserted into the first virus genome comprises a selective marker gene and the step of recovering the infectious modified virions produced by the first host cell comprises a step of infecting a second host cell with those infectious virions under conditions that select for a virus genome expressing the selective marker gene. In a preferred embodiment of this aspect of the invention, expression of the selective marker gene in the second host cell confers on the second host cell resistance to a cytotoxic drug. This drug is present during infection of the second host cell at a level sufficient to select for a virus genome expressing the selective marker gene. In this case the drug selects for a modified virus genome having the inserted selective marker gene and selects against any genome lacking that marker gene (FIG. 5).

In further embodiments of the invention, the method can be used to address safety concerns regarding the storage of viruses such as the variola or smallpox virus. In particular, a virus can be digested with restriction enzymes to render it inactive during storage. The virus can then be re-assembled or reactivated using the method of the invention. In a specific embodiment, the viral fragments can be stored in separate containers and even in separate locations prior to reassembly using the method of the invention.

Accordingly, the present invention provides a method of producing a first recombinant virus comprising:

(a) extracting nucleic acids from a first virus;

(b) preparing fragments of the nucleic acids and separating the fragments into different containers wherein each container will not contain a sufficient number of fragments to prepare an active first virus;

(c) optionally, storing the containers;

(d) providing a host cell that is infected with a second virus;

(e) introducing two or more nucleic acid fragments from at least two different containers into the host cell, wherein said two or more nucleic acid fragments have ends that are capable of being joined;

(f) incubating the host cell under conditions to allow the nucleic acid fragments to recombine and form a recombinant virus; and

(g) recovering the recombinant virus.

The container can be any vessel that is suitable for storing nucleic acids including test tubes and microwell plates. Preferably, at least two containers are used.

The first virus can be any virus, preferably from the family Poxviridae, more preferably from the genus orthopoxvirus, most preferably from the species variola virus or smallpox virus.

(ii) Kits

The reagents suitable for carrying out the methods of the invention may be packaged into convenient kits providing the necessary materials, packaged into suitable containers. For example the reagents may include a host cell and a second virus strain suitable for packaging the modified first viral genome into infectious virions.

In embodiments of the present invention, the kit may further include a DNA sequence comprising the first viral genome, restriction enzymes to cut the first viral genome at unique site(s) and/or reagents to perform the PCR reaction.

The kit may further include a cell line suitable for isolating the reactivated modified first viral genome. In an embodiment of the present invention the cell line comprises BSC-40 cells.

With particular regard to assay systems packaged in “kit” form, it is preferred that assay components be packaged in separate containers, with each container including a sufficient quantity of reagent for at least one assay to be conducted. A preferred kit is typically provided as an enclosure (package) comprising one or more containers for the within-described reagents.

The reagents as described herein may be provided in solution, as a liquid dispersion or as a substantially dry powder, e.g., in lyophilized form. Usually, the reagents are packaged under an inert atmosphere.

Printed instructions providing guidance in the use of the packaged reagent(s) may also be included, in various preferred embodiments. The term “instructions” or “instructions for use” typically includes a tangible expression describing the reagent concentration or at least one assay method parameter, such as the relative amounts of reagent and sample to be admixed, maintenance time periods for reagent/sample admixtures, temperature, buffer conditions, and the like. The instruction may also include guidance on the proper design of PCR primers to allow the addition of homologous sequences onto the PCR amplified fragments.

In another embodiment, the cloning kit further comprises a first host cell and a second (helper) virus suitable for packaging the modified viral genome into infectious virions.

The following non-limiting examples are illustrative of the present invention:

### EXAMPLES

### Methods and Materials

Virus and Cell Culture

Vaccinia virus strain WR, SFV strain Kasza, myxoma virus strain Lausanne and rabbit SIRC cells were originally obtained from the American Type Culture Collection. Vaccinia virus strain Copenhagen was obtained from Dr. N. Scollard (Aventis-Pasteur Canada), vaccinia strain VTF7.5 from Dr. P. Traktman (Medical College of Wisconsin) and modified vaccinia strain Ankara bearing a lacZ insertion [MVA LZ (18)] from Dr. J. Bramson (McMaster University). BSC-40 cells were obtained from Dr. E. Niles (SUNY Buffalo), BGMK cells from Dr. G. McFadden (University of Western Ontario), and BHK-21 cells from Dr. Bramson. All cells were propagated at 37° C. in 5% CO₂ in Minimum Essential Medium supplemented with L-glutamine, non-essential amino acids, antibiotics and antimycotics, and 5-10% fetal calf serum (Cansera). SFV and myxoma viruses were propagated on SIRC cells and most vaccinia on BSC-40 cells. MVA LZ was propagated on BHK-21 cells.

Recombinant Virus Construction

Vaccinia strain XY-I-SceIVV was constructed using standard methods. Briefly, pTM3 (3, 11) was digested with NcoI and XhoI and the excised polylinker replaced with a 44 bp oligonucleotide adaptor encoding the underlined I-Scel site (5′ CAT-GGT-AGG-GAT-MC-AGG-GTA-ATG-TGC-ACC-ATC-ACC-ACC-ACC-AC 3′ (SEQ ID NO:1) and 5′ TCG-AGT-GGT-GGT-GGT-GAT-GGT-GCA-CAT-TAC-CCT-GTT-ATC-CCT-AC 3′ (SEQ ID NO:2)). The resulting plasmid (pXY-I-Scel) was purified, partially sequenced to confirm the insert structure, and calcium phosphate used to transfect the DNA into vaccinia virus infected BSC-40 cells. Recombinant gpt⁺ viruses were passaged three times and plaque purified twice using mycophenolic acid selection. Southern blots were used to confirm the structure of the selected recombinant virus and to confirm that the introduced site can be cut by I-Scel.

Virus Reactivation Assays and DNA Transfection Methods—

BGMK cells were grown to near confluency in 60 mm dishes and then infected with SFV at a multiplicity of infection of 1-2 for 1 hr at room temperature in 0.5 mL of phosphate buffered saline (PBS). The buffer was replaced with 3 mL of warmed growth medium and the cells returned to the incubator for another hour. Lipofectamine complexes were prepared by mixing 2-5 μg of vaccinia DNA, in 0.5 mL OptiMEM medium, with diluted LipofectAmine LF2000 reagent (6-15 μL LipofectAmine plus 0.5 mL of OptiMEM medium). The mixture was incubated for 20 min at room temperature and then 1 mL was added to each dish of cells, and incubated another 4 hr at 37° C. in a CO₂ incubator. The transfection solution was replaced with 5 mL of fresh growth medium and the cells cultured another 3-4 days at 37° C. Virus particles were recovered by scraping the cells into the culture medium and subjecting the mix to three cycles of freeze and thaw. This crude extract was diluted 10-to-105-fold in PBS and plated on BSC-40 cells to recover vaccinia virus. Plaques were stained with a solution containing either X-gal, to detect recombinant b-galactosidase activity, or with Giemsa or crystal violet stain, to titrate total virus.

Other DNAs

Vaccinia virus particles were purified by sedimentation through sucrose gradients and then the DNA was recovered and purified by proteinase K digestion, phenol extraction and ethanol precipitation. A commercial pulsed field gel electrophoresis system and 1% agarose gels were used as directed by the manufacturer (BioRad) to size fractionate vaccinia DNAs. Gene targeting experiments used a number of different b-galactosidase gene cassettes prepared using the PCR and several different primer pairs. A high fidelity DNA polymerase (“Expand High Fidelity PCR System”, Roche) was used as directed by the manufacturer. The template was plasmid pTKZ-1, which encodes the Escherichia coli β-galactosidase gene regulated by a vaccinia virus 7.5S promoter (17). DNAs designed to target the endogenous NotI site in wild-type vaccinia virus, were prepared using the two 37-mer primers pTKZ1-LacZNotI18-A (5′-ACA-CCG-ACG-ATG-GCG-GCC-CTT-AAA-AAT-GGA-TGT-TGT-G-3′) (SEQ ID NO:3) and pTKZ1-LacZNotI18-B (5′ TTC-GTG-TCT-GTG-GCG-GCC-CCT-CM-MT-ACA-TM-ACG-G 3′) (SEQ ID NO:4). This created a targeting cassette sharing 2×18 base pairs of flanking homology with NotI-cut virus. To prepare inserts targeting the I-Scel site in virus XY-I-SceIVV, the inventors PCR amplified the insert using the 37-mer primers pTKZ1-LacZ-A (5′ GAT-MT-ACC-ATG-GTA-GGG-CTT-AAA-MT-GGA-TGT-TGT-G 3′) (SEQ ID NO:5) and pTKZ1-LacZ-B (5′ ATG-GTG-CAC-ATT-ACC-CTG-CCT-CM-MT-ACA-TM-ACG-G 3′) (SEQ ID NO:6) or the 69-mer primers pTKZ1-LacZ-A50 (5′ CCA-CGG-GGA-CGT-GGT-TTT-CCT-TTG-AAA-MC-ACG-ATA-ATA-CCA-TGG-TAG-GGC-TTA-AAA-ATG-GAT-GTT-GTG 3′) (SEQ ID NO:7) and pTKZ1-LacZ-B50 (5′ TM-TTA-ATT-AGG-CCT-CTC-GAG-TGG-TGG-TGG-TGA-TGG-TGC-ACA-TTA-CCC-TGC-CTC-AAA-ATA-CAT-AAA-CGG 3′) (SEQ ID NO:8). This created DNA cassettes sharing 2×18 (7.5KZ18) or 2×50 (7.5KZ50) base pairs of flanking homology with SceI cut XY-I-SceIVV, respectively. A similar approach was used to target an open reading frame encoding enhanced green fluorescent protein (GFP) to the same I-Scel locus. In this case the gene was PCR amplified using the primers GFP-SceI20A (5′ ACGAT-MT-ACC-ATG-GTA-GGG-ATG-GTG-AGC-AAG-GGC-GAG-GA 3′) (SEQ ID NO:9) and GFP-SceI2OB (5′ TGATG-GTG-CAC-ATT-ACC-CTG-TTA-CTT-GTA-CAG-CTC-GTC-CA 3′) (SEQ ID NO:10) and a pEGFP-N1 template (Clontech).

In addition to these substrates, a series of long overlapping PCR fragments spanning nearly all of the vaccinia genome were prepared using the primer pairs summarized in Table 1. A number of different thermoresistant DNA polymerases were tested for use in this application, Roche “Expand” long template PCR kits was eventually found to most reliably amplify long PCR fragments. The DNA sequence of vaccinia virus strain Copenhagen (GenBank entry M35027) and a draft sequence of vaccinia strain WR (kindly provided by Dr. B. Moss, National Institutes of Health) were used in primer design work. These and other PCR-amplified DNAs were gel purified and electroeluted before use. Spectrophotometry was used to calculate all of the DNA concentrations prior to transfection.

Confocal Microscopy

The production of GFP by recombinant viruses was detected using a Leica TCS SP2 confocal microscope. BSC-40 cells were cultured on glass slides, co-infected with a mixture of reactivated/recombinant vaccinia virus and a vaccinia virus expressing T7 RNA polymerase (VTF7.5), and imaged 24 hr post-infection. The expression of GFP was detected using epifluorescence while cells were imaged using differential interference contrast (DIC) optics.

### RESULTS

Reactivation of Vaccinia Virus by Shope (Rabbit) Fibroma Virus—

The Leporipoxvirus Shope fibroma virus (SFV) and the Orthopoxvirus vaccinia offers several attractive biological features that simplify the experimental approach that follows. In particular, SFV has a very narrow host range -replicating only rabbit cells and a few selected monkey cells (BGMK). It also grows slowly to modest titers (˜10⁷ PFU/mL) and the minute (˜1 mm) plaques look much like transformed foci. In contrast, vaccinia virus has a much broader host range than SFV, grows rapidly to high titers (˜10⁹ PFU/mL) and produces large and distinctive cytolytic plaques. As with previously described vaccinia/fowlpox systems, these phenotypic properties greatly facilitate the separation and differentiation of mixtures of SFV and vaccinia viruses.

The inventors infected BGMK cells with SFV and two hours later transfected these cells with 2-5 μg of DNA extracted from sucrose gradient purified particles of vaccinia strain XY-I-SceIVV. Three days post-transfection, all of the infectious particles were recovered by cell lysis and replated on a BSC-40 cell line that supports only the growth of vaccinia virus. The resulting stained dishes are shown in FIG. 5. Large amounts of virus were recovered using this strategy (yields ranged up to 10⁷ PFU/dish of transfected cells) and the plaques visually resembled those produced by the parent strain of vaccinia virus.

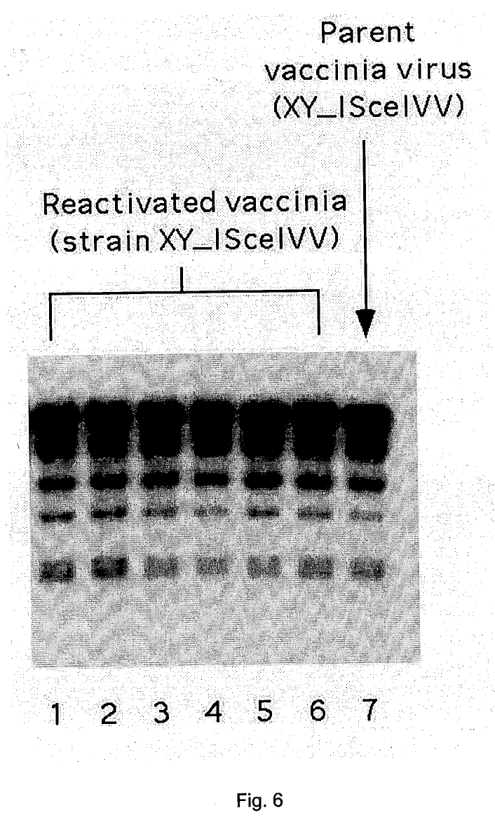

Strain XY-I-SceIVV encodes a gpt selectable marker and the reactivated viruses also plated efficiently in the presence of mycophenolic acid (74% of the plaques recovered in the absence of selection). The limit of sensitivity was <20 PFU/mL, within this experimental constraint no plaques were detected when vaccinia DNA was transfected into uninfected cells, nor were any cytolytic plaque forming particles recovered from cells infected only with SFV. Microscopic inspection of the control dishes also failed to detect any plaques resembling the foci formed by SFV, although the inventors could not preclude the possibility that SFV establishes an abortive infection in BSC-40 cells. To prove that the method produces bona fide vaccinia viruses, the inventors plaque purified several independent virus isolates, extracted virus DNA and used Southern blots to compared the HindIII fingerprint of each isolate with that of the parent vaccinia strain XY-I-SceIVV and its precursor strain vaccinia WR. FIG. 6 illustrates one such Southern blot; all of the rescued viruses appeared identical to the parent strain at this level of resolution.

Reciprocal Reactivation of Leporipoxviruses

The inventors also tested whether the reciprocal experiment would work, that is can an Orthopoxvirus reactivate a Leporipoxvirus?The inventors took advantage of the narrow host range of modified vaccinia virus strain Ankara to test whether MVA could reactivate myxoma virus. (Myxoma was used in these experiments because it produces, more easily visualized and accurately titered plaques than does SFV.) Preliminary tests showed that both viruses can replicate efficiently on hamster BHK-21 or monkey BGMK cells, but only myxoma virus produces plaques on rabbit SIRC cells. The inventors infected BGMK cells with a lacZ⁺ derivative of MVA [MVA LZ (18)] and then transfected the cells with wild-type myxoma virus DNA. Four days latter the resulting virus were recovered and plated on SIRC cells. Virus were recovered with yields of ˜300 PFU/μg of transfected DNA and none of these plaques stained positively for the lacZ marker characteristic of MVA LZ or lacZ⁺ intertypic recombinants. Thus it would seem that although the reaction is less efficient, if one uses the appropriate selection strategy an Orthopoxvirus can reactivate a Leporipoxvirus.

Genetic Recombination is Associated with Virus Reactivation

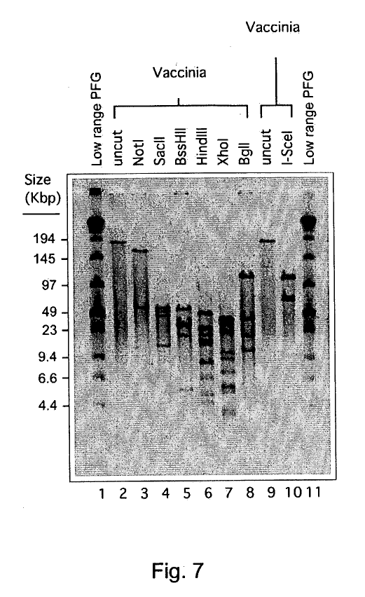

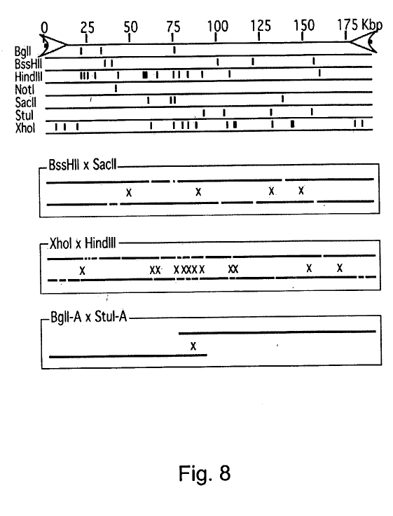

The vaccinia genome spans 196 kbp and no special efforts were made to avoid shearing viral DNA during the process of DNA extraction. Pulsed-field gels showed that the double-stranded DNA used in these experiments contained the expected distribution of broken molecules ranging in size from <10 kbp to near full length (FIG. 7, lane 2). The inventors have previously shown (21) that poxvirus-infected cells catalyze high-frequency recombination of transfected DNAs using a single-strand annealing mechanism, and presumed that SFV-infected cells catalyze recombinational repair of this sheared vaccinia DNA in much the same way in reactivation reactions. To examine this question in more detail, the inventors separately digested purified wild-type vaccinia virus DNA with BssHII and SacII and examined the ability of SFV-infected cells to reconstruct intact genomes and live viruses from these linearized fragments. Pulsed field gels showed that these enzymes cut vaccinia strain WR DNA to completion (FIG. 7) and the restriction fragments, with some strain-specific exceptions, closely matched that predicted by computational methods (FIG. 8). When these DNAs were transfected separately into SFV infected BGMK cells, they produced no recombinant vaccinia viruses detectable by plating on BSC-40 cells (<20 PFU/dish). However, cotransfecting a mixture of SacII and BssHII-cut DNAs into SFV-infected cells permitted the production of infectious vaccinia particles at levels essentially identical to the control, uncut, reaction efficiency (2.5×10⁵ versus 2.6×10⁵ PFU/dish). Similarly, co-transfecting SFV-infected cells with an equimolar mixture of two large gel-purified BgII-A and StuI-A restriction fragments (FIG. 8) also permitted the recovery of recombinant viruses (2×10³ PFU/dish). There are nevertheless limits to these reactions. Attempts to reconstruct vaccinia from a mixture of HindIII and XhoI cut molecules were unsuccessful, suggesting that such enzymes probably cut too frequently or too close to each other to preclude the reassembly of intact vaccinia genomes by SFV-infected cells.

Production of Recombinant Viruses by Targeted Double-Strand Break Repair

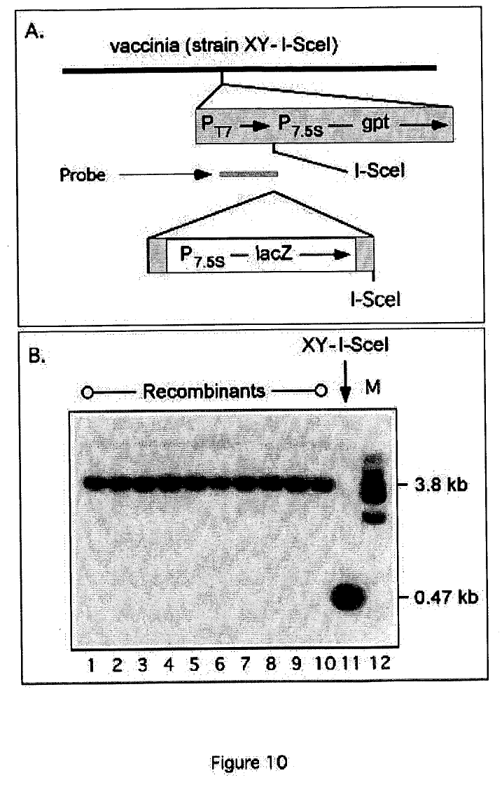

This reaction can be exploited to simplify the construction and recovery of recombinant vaccinia viruses without plasmid cloning or DNA ligation reactions. Vaccinia virus was modified using standard molecular biological and plasmid-by-virus recombination methods to incorporate an I-Scel site and E. coli gpt selectable marker into the thymidine kinase gene locus (strain XY-I-SceI, FIG. 10). Virus DNA was then isolated from purified XY-I-SceI particles, digested with I-Scel and co-transfected along with a 20-fold mol excess of a PCR-amplified β-galactosidase gene cassette into SFV-infected cells (FIG. 10). In this case, the β-galactosidase gene was placed under the regulation of a 7.5S promoter and the PCR amplicon incorporated 2×18 bp of end sequences identical to sequences flanking the recombinant I-Scel site.

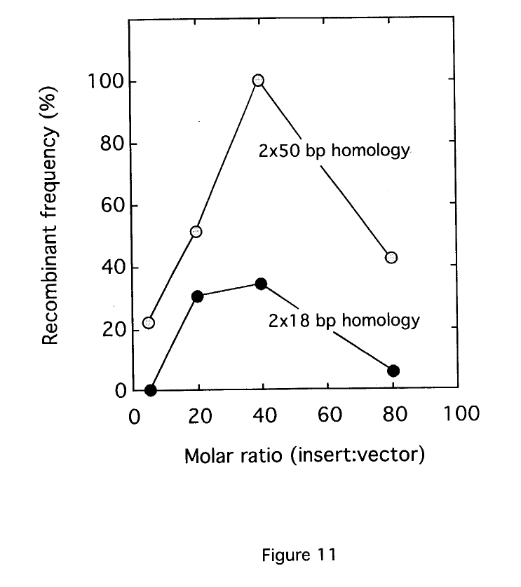

X-gal staining showed that this approach can produce about 30% recombinant viruses and Southern blots confirmed that all of the putative recombinants tested (10/10) arose through the expected targeted recombination between the β-galactosidase gene and the I-SceI cleavage site (FIG. 10). Subsequent experiments showed the frequency of recombinant production is enhanced by increasing the ratio of insert to virus vector and by increasing the length of terminal homology. Cells co-transfected with I-SceI-cut vaccinia virus DNA, and a 40-fold excess of PCR-amplified DNA, produced 100% lacZ⁺ recombinant viruses when the homology was increased to 2×50 bp (FIG. 11). The inventors also confirmed that these results are not just specific for I-Scel cut vaccinia DNA. NotI cuts vaccinia virus strain WR only once in non-essential sequences (10). Similar yields of recombinant virus (4×10⁴ PFU/μg, 22% recombinants) were obtained when lacZ-encoding PCR amplicons, prepared using primers that added 2×18 nt of sequence homologous to that flanking the NotI site, were cotransfected into SFV-infected cells along with NotI-cut vaccinia DNA.

The production of lacZ⁺ viruses need not have involved homology, since non-homologous end-joining reactions could serve the same purpose and Southern blots would not be capable of discriminating between these two types of reactions. The inventors tested the requirement for homology using a combination of I-SceI-cut virus and the PCR amplicon originally designed to recombine with NotI-cut viral DNA. Such a combination of virus and DNA share no end-sequence homology beyond a few chance nucleotides. Co-transfecting this mixture of I-SceI-cut vaccinia virus and PCR amplified DNAs into SFV-infected cells yielded significant numbers of virus (5×10⁵ PFU/μg), possibly by direct ligation, but only 0.08% were lacZ⁺ recombinants. This low frequency of non-homologous recombination is thus very similar to that previously observed in vaccinia-infected cells, using transfected fragments of luciferase-encoding DNA (21).

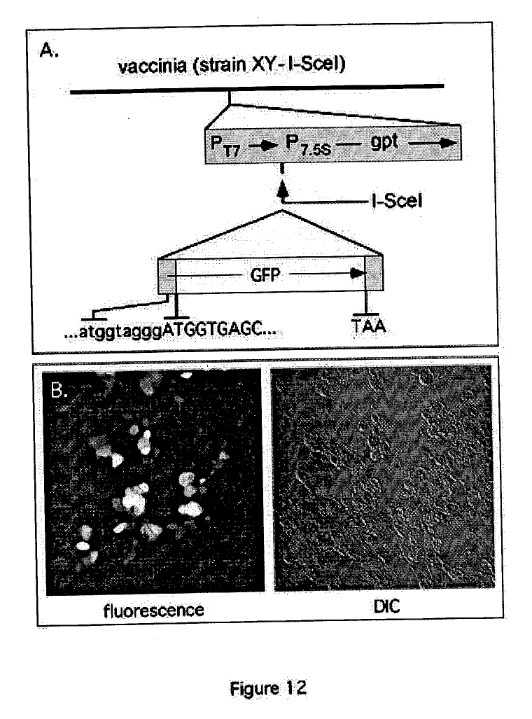

Because the I-SceI site is preceded by a T7 promoter and internal ribosome entry site derived from plasmid pTM3 (3, 11), the virus vector used in these reactions can also be used for the direct cloning and expression of recombinant proteins. DNA was extracted from vaccinia strain XY-I-Scel, digested with I-Scel, and cotransfected into SFV-infected cells along with a 760 bp promoterless DNA fragment encoding a green fluorescent protein (GFP) open reading frame. Two 20 nt regions of homology permitted a recombination reaction that was expected to place the GFP gene under the regulation of the T7 promoter (FIG. 12A). Three days post-transfection, the resulting mixture of recombinant and non-recombinant viruses was recovered and subsequently co-cultivated for another 24 hr on glass cover slips along with a helper virus expressing T7 RNA polymerase (5). Fluorescence microscopy was used to identify which infected cells produced recombinant green fluorescent protein. A significant portion (perhaps one-third) of the infected cells expressed GFP under these conditions (FIG. 12B).

Targeted Deletion of a Vaccinia Virus Restriction Fragment—

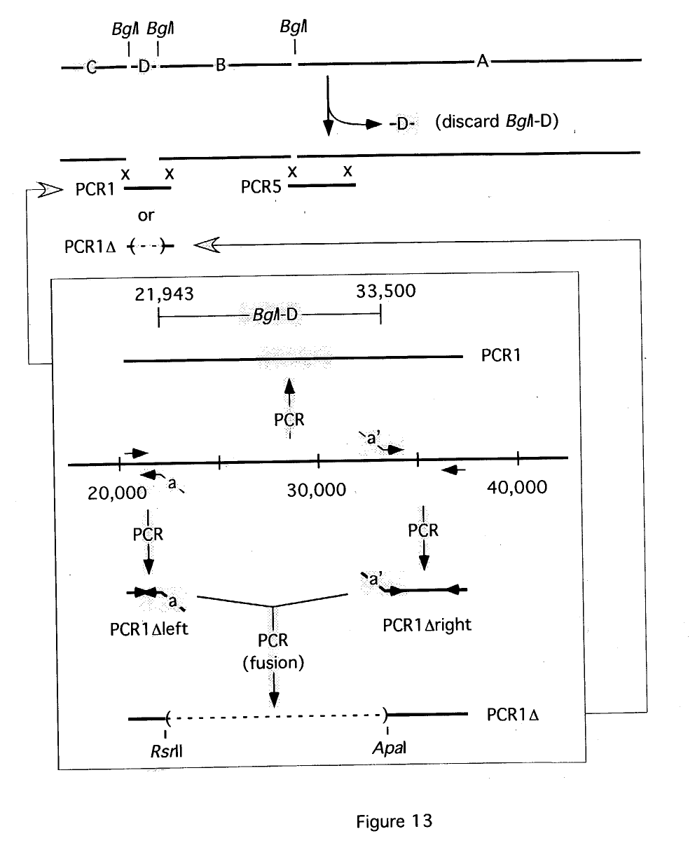

The efficiency of SFV-catalyzed reactivation methods suggested that the approach might also be used to assemble other modified forms of vaccinia genomes. To test this hypothesis, the inventors investigated whether an 11.5-kbp fragment of the vaccinia genome could be deleted in a single step using a specially designed PCR amplicon. The experiment involved first digesting vaccinia virus DNA with BglI, and then recovering the three largest DNA fragments from an agarose gel. The 11.5 kbp BglI-D fragment discarded at this stage has been shown previously to lack any genes essential for replication in culture (13). Four PCR primers, a vaccinia DNA template, two ordinary PCR reactions, and a subsequent PCR fragment fusion reaction were then used to prepare a 3.6 kbp linker DNA sharing end sequence homology with the two fragments flanking the missing BglI-D fragment, but omitting nucleotides 21943 to 33500 (FIG. 13). This linker DNA (PCR1Δ) was then transfected into SFV-infected cells along with the other three BglI restriction fragments and a large PCR-amplified splice fragment (PCR5). PCR5 DNA was added to direct the recombinational repair of the double-stranded break separating BglI-A and BglI-B fragments (FIG. 13). Reactivated virus were then recovered, plaque purified, and characterized using the PCR and Southern blots (data not shown). In these experiments, the yield of reactivated virus was 3.8×10³ PFU/μg and 100% of the virus (10/10) encoded a deletion of the expected bases. This yield of virus was very similar to that obtained by transfecting the three BglI restriction fragments into SFV-infected cells along with fragment PCR5 and a PCR fragment (PCR1) encoding all of the sequences deleted in PCR1 D.

It should be noted that the virus reactivated in the control reactions from mixtures of three BglI restriction fragments, plus PCR1 and PCR5 DNA fragments, are indistinguishable from the parental virus (vaccinia strain WR). This is because all of the DNAs were prepared using vaccinia WR reagents. To confirm that the genetic information incorporated between nucleotides 21943 and 33500 actually derived from PCR1, and not from a contaminating BglI-D fragment, the inventors also prepared a PCR1 fragment using vaccinia strain Copenhagen DNA as the template. All of the virus reactivated from cells transfected with this PCR1_{cop} DNA, plus WR-derived BglI and PCR5 fragments, bore an Xbal polymorphism indicative of the presence of BglI-D sequences originating in vaccinia strain Copenhagen (8 of 8 viruses tested, data not shown). Besides demonstrating the purity of the mixture of strain WR-derived BglI-A, BglI-B, and BglI-C fragments, this result illustrates how the method can be used to more precisely control the assembly of recombinant viruses from different viral strains.

In these experiments, the inventors should also note that the PCR1Δ linker fragment was assembled from two separate DNAs, each encoding one of the two sequences homologous to those found flanking the BglI-D fragment. The assembly was accomplished using additional homologous sequences incorporated into the two central primers, and an in vitro PCR fusion reaction, to combine the 3.3 kbp (PCR1Δ-left) and 0.4 kbp (PCR1Δ-right) fragments into a single 3.6 kbp linker (PCR1Δ, FIG. 13). This step proved to be unnecessary, because deletion virus could also be recovered from SFV-infected cells that had been transfected with PCR1Δ-left, PCR1Δ-right, three BglI restriction fragments, and PCR5. However, requiring the additional recombinational exchange between DNAs sharing 30 nt of sequence homology, may have been responsible for reducing the yield of reactivated virus about five-fold (from 4×10³ to 8×10² PFU/μg).

Recombinational Substitution of Essential Portions of the Vaccinia Genome Using Large PCR-Amplicons—

The studies described above, showed that one can delete the BglI-D fragment and rescue the deficiency in reactivated viruses using a large PCR-amplified homolog. However, this is not a very rigorous test of the method since the inventors used only a single fragment of DNA and BglI-D encodes no genes essential for virus replication. As a more demanding test of the approach, the inventors examined whether portions of the virus encoding genes essential for growth in culture could also be PCR amplified and rescued into viable virus.

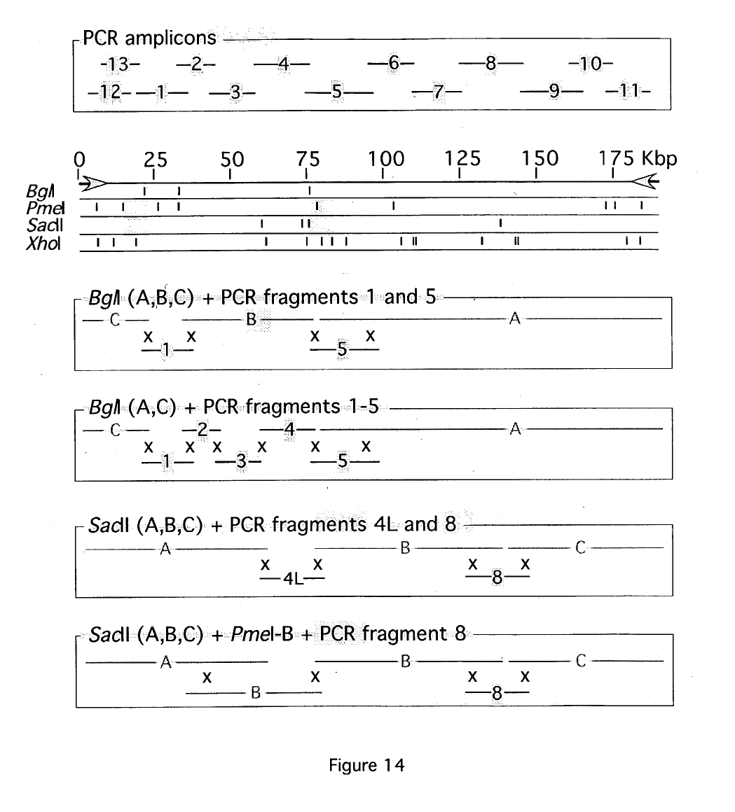

The first study examined whether a nearly complete set of overlapping PCR products could be assembled into a reactivated virus. The inventors used “Expand” long range PCR reactions to amplify a series of 12-22 Kbp overlapping fragments spanning most of the vaccinia virus genome (FIG. 14). These fragments included the PCR1 and PCR5 fragments used previously. The lengths of the overlaps between different PCR fragments ranged from 0.3 to 9.3 Kbp and were randomly determined by the manner in which a primer design program (Oligo 6) identified suitable primers. The inventors did not try to amplify DNA located at the immediate ends of the genome because of anticipated difficulties using PCR to reproduce such telomeric features as hairpins and mismatched bases. Instead, vaccinia genomic DNA was digested with Xhol and the resulting ˜5 kbp restriction fragments isolated from agarose gels. All of these DNA fragments were combined in the appropriate molar ratios, co-transfected into SFV-infected BGMK cells, and any resulting virus rescued by replating on BSC-40 cells.

It was of some concern that all of the DNAs used in these experiments had been purified from agarose gels, because this method can introduce contaminants into DNA substrates. To show that there were no inhibitory contaminants present, the inventors added Xhol-cut vaccinia virus DNA to the mixture and co-transfected this pool of substrates into SFV-infected cells. This mix of natural and synthetic DNA fragments permitted recovery of virus with a yield of ˜2×10³ PFU/μg.

To gain some understanding as to what other factor(s) might have prevented these experiments from working, the inventors examined whether progressively less complex mixtures of natural and PCR-amplified vaccinia virus DNAs could be recombined and reactivated in SFV-infected cells. Noting that a mixture of PCR1 and PCR5 fragments, along with BglI-A, -B, and -C restriction fragments, permitted recovery of reactivated virus, the inventors tested whether virus could also be rescued from a combination of just the BglI-A and BglI-C restriction fragments plus PCR fragments 1-to-5 (FIG. 14). Again, no reactivated viruses were recovered using this strategy. Finally, the inventors further simplified the experiment so that only a single large and yet essential PCR fragment had to be rescued into the vaccinia genome. Several different regions of the vaccinia genome were examined and the inventors were able to reproducibly recover recombinant and reactivated virus using at least one particular combination of natural and PCR-amplified DNA.

These studies used the three largest vaccinia virus SaclI restriction fragments and PCR fragments 4L and 8 (FIG. 14). PCR4L (a slightly larger derivative of PCR4) shared 3.3 and 2.5 Kbp of flanking sequence homology with the adjacent SaclI fragments and spanned the genetic interval encompassing genes I13L to L4R. It thus encoded many genes known to be essential for viral growth and assembly (6, 7, 14, 20, 22). PCR8 served only as a recombinational bridge between SaclI-B and SaclI-C fragments (FIG. 14). When SFV-infected BGMK cells were transfected with this DNA mixture, the inventors obtained yields of recombinant virus that were essentially identical to those obtained when a control Pmel-B restriction fragment was used instead of PCR4L (8.5×10⁵ versus 8.2×10⁵ PFU/μg, respectively).

Southern blots were later used to confirm that all (10/10) of the virus recovered and tested were genetic hybrids. To show this, the inventors assembled a recombinant virus using a heterologous combination of WR SaclI restriction fragments and Copenhagen “templated” PCR4L_{cop}DNA, and used restriction fragment polymorphisms to identify the origins of different parts of the resulting virus. A probe targeting SaclI-D sequences detected a HincII-site polymorphism in the reactivated viruses characteristic of strain Copenhagen, while a probe targeting the BglI-D region (FIG. 14) detected an Xbal-site polymorphism characteristic of strain WR (data not shown). The inventors concluded that one can rearrange essential portions of the vaccinia genome using these methods, but preferably only a single amplicon at a time.

### DISCUSSION

These experiments show that SFV can be used to rescue and reactivate vaccinia virus in cells transfected with vaccinia DNA. Importantly, this seems to be by far the most efficient in vitro heterologous poxvirus reactivation reaction described to date, and this contention is supported by direct comparisons. These show that SFV-infected cells yield ˜100-fold more reactivated vaccinia virus than do fowipox-infected cells (M. Merchlinsky, personal communication). This increase in efficiency offers significant experimental advantages, but the numbers still suggest that only a small proportion of input genomes contribute to the pool of reactivated viruses that one can eventually recover from SFV-infected cells. Perhaps this is not too surprising because, during the early steps in the process of virus rescue, a mixture of virus proteins would be expected to arise that might well interfere with the activity of multi-component protein complexes or the assembly of virus capsids.

Mixed infections of Orthopoxviruses, Leporipoxviruses and Avipoxviruses are thus either able to segregate orthologous proteins into properly distinct protein complexes, or the architecture of these complexes is sufficiently flexible to accommodate proteins typically sharing only 60-80% amino acid identity. The observation that SFV seems to reactivate vaccinia much better than does fowlpox virus, suggests that the later process may operate under these experimental conditions since SFV proteins might be more compatible with vaccinia proteins given the closer evolutionary relationship. SFV-infected cells also catalyze very high levels of non-specific DNA replication (1) and recombination (4, 12) and these reactions may be another factor contributing to the efficiency of the overall process by more efficiently amplifying and repairing transfected vaccinia genomes.

One advantage of using fowlpox helper viruses is that the genetic distances, which separate Avipoxviruses from Orthopoxviruses, minimize the risk of mixed infections producing intertypic virus recombinants. Leporipoxviruses and Orthopoxviruses also appear to have been sufficiently isolated by evolutionary processes to prevent SFV from recombining with vaccinia virus. All of the viruses that the inventors have rescued to date seem to grow normally and, although the inventors have not pursued an exhaustive screen for intertypic recombinants, no such viruses were detected using either Southern blots (vaccinia) or genetic methods (myxoma).

This failure to recover intertypic recombinants probably depends upon two favorable factors. First, hybrid viruses would probably exhibit growth deficiencies of varying severity and that would reduce their abundance in mixed populations of replicating viruses. Second, when one compares DNA sequences, about ¼ of the bases differ between even the most closely related virus genes (probably SFV S068R and vaccinia J6R) and one cannot detect even this limited homology using Southern blots (FIG. 6). This is probably insufficient sequence identity to permit efficient recombination and, collectively these two constraints would compromise the recovery of intertypic recombinant viruses. The inventors believe that, as a method of genetic isolation, using helper viruses like fowlpox and SFV to reactivate Orthopoxviruses is preferred to using homologous psoralen-inactivated Orthopoxviruses (19). Heterologous helper viruses seem to be genetically inert while chemically-inactivated viruses could contribute heavily damaged DNAs to a pool of molecules interacting in a highly recombinogenic environment.

Several practical uses for the method have been identified in this study, which exploit the high frequency recombination and non-specific DNA replication systems we've previously characterized. In particular one can utilize fortuitously located restriction sites and PCR-generated linker fragments to create targeted deletions of non-essential portions of poxvirus genomes. One could presumably continue using this process in a stepwise manner, by taking further advantage of pre-existing as well as newly introduced restriction sites to create a succession of progressively smaller viruses. Appropriately modified viruses can also be used to facilitate the conditional expression of recombinant proteins. The production of recombinants is most efficient when rather long patches of flanking homology are used to target the insert into the double-stranded break (2×50 bp, FIG. 11), but even 2×18 bp patches of homology can yield recombinants at frequencies of up to 30%. In this regard the effect of homology length on reaction efficiency are qualitatively similar to those we've previously characterized in vaccinia-infected cells (12, 21) although the different selection methods render absolute comparisons difficult. SFV-promoted recombination and reactivation reactions are sufficiently efficient that one can directly detect the expression of vaccinia-encoded recombinant green fluorescent protein without further selection, propagation, or plaque purification of the recombinant virus (FIG. 12).

Despite the high recombination frequencies detectible in SFV- and vaccinia-infected cells, it seems likely that a “numbers game” ultimately places practical limits on the capacity of these systems to generate recombinant viruses. These limits are of little concern where a simple double strand break repair reaction is used to insert one piece of DNA into a cut vector using reactions of the type illustrated in FIGS. 10 and 12. However, as the number of exchanges increases, the overall yield of reactivated virus is expected to decrease in a manner that depends upon the efficiency of each component recombination reaction. This is best illustrated by considering the impact of each additional recombination step occurring with an efficiency near 50% verses only 20%. The overall yield of virus is crudely expected to follow the relationship N=N_{o·E}^{X} where N= overall yield of virus (PFU/μg), Nₒ=maximal yield possible using intact transfected DNA, E= average efficiency of each component recombination event, and x= number of exchanges. With a typical maximal yield of approximately Nₒ₌10⁶ PFU/μg and the lowest practical yield N=1 PFU/μg, solving for “x” suggests that virus should be recoverable if the number of exchanges ranges from eight (E=0.2) to twenty (E=0.5).

These values do seem to be useful working limits with this system. For example, very high yields of virus were obtained using mixtures of BssHII-and SaclI-cut vaccinia DNA (FIG. 8) in a reaction requiring only four exchanges and involving extensive (i.e. efficiently recombined) overlaps. Conversely, virus could not, be recovered from cells transfected with a mixture of XhoI- and HindIII-cut vaccinia DNA. In this situation, at least twelve exchanges are required and some of the short overlaps between fragments (as little as 0.2 Kbp) might also be expected to reduce the average recombination efficiency.

A rather surprising feature of this process is that it can be used to reactivate vaccinia viruses from transfected mixtures of virus restriction fragments and large PCR-amplified portions of the virus genome. It is surprising, because it is expected that viruses produced by this method would encode multiple new mutations due the poor fidelity of the DNA polymerases used in the PCR. These error frequencies vary from 2.6×10⁻⁵ (for Taq polymerase) to 8.5×10⁻⁶ (Roche “high fidelity” PCR system) with the Roche “long template” PCR systems the inventors are using exhibiting an accuracy that probably falls somewhere between these two bounds. If one used twenty PCR cycles to create a pool of ˜17 kbp PCR products, each DNA would then bear an average of 3 or 13 mutations per molecule if these DNAs were amplified using high-fidelity or Taq polymerases, respectively. Only ˜5% of the 17 kbp molecules amplified using high fidelity proofreading enzymes would be expected to be free of errors and essentially none of the DNAs amplified using Taq polymerase would be error free. Most of these mutations would be silent and so these errors might not be enough to prevent the recovery of recombinant viruses using a single large PCR amplicon encoding numerous essential virus genes. However, it becomes more and more unlikely that one could reactivate a virus from mutation-free PCR amplicons, as the number of such fragments increases. This fact may explain why one cannot reactivate virus from multiple pieces of PCR-amplified DNAs and suggests that even the most efficient of poxvirus reactivation methods couldn't provide a facile route for reactivating Orthopoxviruses if the only available source of virus DNA was PCR-amplified or chemically synthesized materials.

In conclusion, the inventors have shown that the DNA replication and recombination systems found in cells infected with replicating poxviruses (1, 12), probably also play an important role in catalyzing virus reactivation reactions. The unusually hyper-recombinogenic environment created in SFV-infected cells can also be exploited to provide a simple method for rearranging the structure of poxvirus genomes and might ultimately even provide a novel way of securely archiving Orthopoxviruses in an inert form. One could envision purifying the virus DNA, cutting it with different restriction enzymes, and storing different digests in separate locations. This process would address public concerns about the storage of variola virus, by rendering the stocks non-infectious and difficult to reactivate unless one had access to both pools of DNA.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

PCR primers used to amplify overlapping fragments
of the vaccinia virus genome.

| | | | | |
|---|---|---|---|---|
| | Size | | | Position |
| Amplicon | (Kbp) | Primer ID | Primer sequence | (WR)¹ |

| | | | | | |
|---|---|---|---|---|---|
| PCR12 | 11.9 | 25VV5533U18 | AGTTAGTTCCGACGTTGA | (SEQ ID NO:11) | 4,900 |
| | | 26VV19848L21 | TATTTGTTGGCTCAGTATGAC | (SEQ ID NO:12) | 16,791 |
| PCR13 | 11.9 | 29VV14300U22 | TATCAGATTATGCGGTCCAGAG | (SEQ ID NO:13) | 7,458 |
| | | 30VV22471L21 | TGTACTATTCCGTCACGACCC | (SEQ ID NO:14) | 19,411 |
| PCR1 | 15.1 | 31VV20807U24 | AGCAAGTAGATGATGAGGAACCAG | (SEQ ID NO:15) | 18,727 |
| | | 32 VV36885L22 | AGGCAGAGGCATCATTTITGGAC | (SEQ ID NO:16) | 33,836 |
| PCR2 | 18.2 | 3VV28266U18 | TTAGTTATTTCGGCATCA | (SEQ ID NQ:17) | 25,217 |
| | | 4VV46527L21 | TTAGTATTTCTACGGGTGTTC | (SEQ ID NO:18) | 43,416 |
| PCR3 | 17.3 | 5VV44435U21 | AGAATATCCCAATAGGTGTTC | (SEQ ID NO:19) | 41,306 |
| | | 6VV61698L20 | CTGTTATTATCGACGAGGAC | (SEQ ID NO:20) | 58,586 |
| PCR4 | 18.6 | 7VV61397U21 | CATTATCTATATGTGCGAGAA | (SEQ ID NO:21) | 58,266 |
| | | 8VV80029L17 | TGACGGGAACAGTAGAA | (SEQ ID NO:22) | 76,914 |
| PCR4L | 21.3 | 7VV61397U21 | CATTATCTATATGTGCGAGAA | (SEQ ID NO:23) | 58,266 |
| | | 8VV79532L29 | GATAACCATGTTCTTATTCTTTCTCCTAC | (SEQ ID NO:24) | 79,532 |
| PCR5 | 19.8 | 9VV78408U18 | AAATGTAGACTCGACGGA | (SEQ ID NO:25) | 75,277 |
| | | 10VV98171L21 | ATAACATATCGACGACTTCAC | (SEQ ID NO:26) | 95,046 |
| PCR6 | 16.51 | 1VV96083U20 | CATAGAAATAAGTCCCGATG | (SEQ ID NO:27) | 92,938 |
| | | 12VV112600L21 | ATGATATTTCTATTGGCCTAA | (SEQ ID NO:28) | 109,475 |
| PCR7 | 17.9 | 13VV111111U19 | AGATCGCTTTCTGGTAACA | (SEQ ID NO:29) | 107,972 |
| | | 14VV129024L21 | TTGCCTCTTACTAGCTTAGTT | (SEQ ID NO:30) | 125,916 |
| PCR8 | 22.3 | 15VV128103V20 | AAGTAGACATAGCCGGTTTC | (SEQ ID NO:31) | 124,975 |
| | | 16VV146278L21 | GTTTATCTTTACGGGCATTAC | (SEQ ID NO:32) | 147,319 |
| PCR9 | 19.2 | 17VV145376U21 | ATGTCCTCTGCCAAGTACATA | (SEQ ID NO:33) | 146,382 |
| | | 18VV164550L20 | AGTACATTATTCACGCTGTC | (SEQ ID NO:34) | 165,581 |
| PCR10 | 15.3 | 19VV159718U21 | TATATTCTTTCAACCGCTGAT | (SEQ ID NO:35) | 160,733 |
| | | 20VV175026L19 | AACCGGGATGTAATAACAC | (SEQ ID NO:36) | 176,016 |
| PCR11 | 20.5 | 23VV169321U21 | TGCCATTATGATAAGTACCCT | (SEQ ID NO:37) | 170,316 |
| | | 24VV187184L21 | TGTCTTTCTCTTCTTCGCTAC | (SEQ ID NO:38) | 190,831 |

¹Except for primer 8VV79532L29, the primer ID specifies the position within the vaccinia (Copenhagen) genome. For example, the 5′-end of primer 25VV5533U18 maps to nucleotide position 5,533. Primer 8VV79532L29 is located at position 82,662 in strain Copenhagen.

### CITATIONS FOR REFERENCES REFERRED TO IN THE SPECIFICATION

1. DeLange, A. M., and G. McFadden. 1986. Sequence-nonspecific replication of transfected plasmid DNA in poxvirus-infected cells. Proc Natl Acad Sci USA 83:614-8.

2. Domi, A., and B. Moss. 2002. Cloning the vaccinia virus genome as a bacterial artificial chromosome in Escherichia coli and recovery of infectious virus in mammalian cells. Proc Natl Acad Sci USA 99:12415-20.

3. Elroy-Stein, O., T. R. Fuerst, and B. Moss. 1989. Cap-independent translation of mRNA conferred by encephalomyocarditis virus 5′ sequence improves the performance of the vaccinia virus/bacteriophage T7 hybrid expression system. Proc Natl Acad Sci USA 86:6126-30.

4. Fisher, C., R. J. Parks, M. L. Lauzon, and D. H. Evans. 1991. Heteroduplex DNA formation is associated with replication and recombination in poxvirus-infected cells. Genetics 129:7-18.

5. Fuerst, T. R., P. L. Earl, and B. Moss. 1987. Use of a hybrid vaccinia virus-T7 RNA polymerase system for expression of target genes. Mol Cell Biol 7:2538-44.

6. Gross, C. H., and S. Shuman. 1996. Vaccinia virions lacking the RNA helicase nucleoside triphosphate phosphohydrolase II are defective in early transcription. J Virol 70:8549-57.

7. Hassett, D. E., and R. C. Condit. 1994. Targeted construction of temperature-sensitive mutations in vaccinia virus by replacing clustered charged residues with alanine. Proc Natl Acad Sci USA 91:4554-8.

8. Joklik, W. K., G. M. Woodroofe, I. H. Holmes, and F. Fenner. 1960.

The reactivation of poxviruses. I. Demonstration of the phenomenon and techniques of assay. Virology 11:168-184.

9. Merchlinsky, M., D. Eckert, E. Smith, and M. Zauderer. 1997. Construction and characterization of vaccinia direct ligation vectors. Virology 238:444-51.

10. Merchlinsky, M., and B. Moss. 1992. Introduction of foreign DNA into the vaccinia virus genome by in vitro ligation: recombination-independent selectable cloning vectors. Virology 190:522-6.

11. Moss, B., O. Elroy-Stein, T. Mizukami, W. A. Alexander, and T. R. Fuerst. 1990. Product review. New mammalian expression vectors. Nature 348:91-2.

12. Parks, R. J., and D. H. Evans. 1991. Effect of marker distance and orientation on recombinant formation in poxvirus-infected cells. J Virol 65:1263-72.

13. Perkus, M. E., S. J. Goebel, S. W. Davis, G. P. Johnson, E. K.

Norton, and E. Paoletti. 1991. Deletion of 55 open reading frames from the termini of vaccinia virus. Virology 180:406-10.

14. Rochester, S. C., and P. Traktman. 1998. Characterization of the single-stranded DNA binding protein encoded by the vaccinia virus 13 gene. J Virol 72:2917-26.

15. Sam, C. K., and K. R. Dumbell. 1981. Expression of poxvirus DNA in coinfected cells and marker rescue of thermosensitive mutants by subgenomic fragments of DNA. Ann. Virol. (Inst. Past.) 132:135-150.

16. Scheiflinger, F., F. Dorner, and F. G. Falkner. 1992. Construction of chimeric vaccinia viruses by molecular cloning and packaging. Proc Natl Acad Sci USA 89:9977-81.

17. Spyropoulos, D. D., B. E. Roberts, D. L. Panicali, and L. K. Cohen. 1988. Delineation of the viral products of recombination in vaccinia virus- infected cells. J Virol 62:1046-54.

18. Sutter, G., and B. Moss. 1992. Nonreplicating vaccinia vector efficiently expresses recombinant genes. Proc Natl Acad Sci USA 89:10847-51.

19. Timiryasova, T. M., B. Chen, N. Fodor, and 1. Fodor. 2001. Construction of recombinant vaccinia viruses using PUV-inactivated virus as a helper. Biotechniques 31:534, 536, 538-40.

20. White, C. L., A. S. Weisberg, and B. Moss. 2000. A glutaredoxin, encoded by the G4L gene of vaccinia virus, is essential for virion morphogenesis. J Virol 74:9175-83.

21. Yao, X. D., and D. H. Evans. 2001. Effects of DNA structure and homology length on vaccinia virus recombination. J Virol 75:6923-32.

22. Zhang, Y., J. G. Keck, and B. Moss. 1992. Transcription of viral late genes is dependent on expression of the viral intermediate gene G8R in cells infected with an inducible conditional-lethal mutant vaccinia virus. J Virol 66:6470-9.

We claim:

1. A method of producing a first recombinant virus comprising:
(a) providing a host cell that is infected with a second virus;
(b) introducing two or more nucleic acid fragments from the first virus into the host cell, wherein said two or more nucleic acid fragments have ends that are capable of being joined;
(c) incubating the host cell under conditions to allow the nucleic acid fragments to recombine and form a recombinant virus; and
(d) recovering the recombinant virus.

2. The method according to claim 1, wherein each of the two or more nucleic acid fragments comprises between 10-9000 basepair (bp) of sequence that is homologous to the fragment to which it is to be joined.

3. The method according to claim 1, wherein wherein each of the two or more nucleic acid fragments comprises between 12-100 basepair (bp) of sequence that is homologous to the fragment to which it is to be joined.

4. The method according to claim 1, wherein wherein each of the two or more nucleic acid fragments comprises between 16-20 basepair (bp) of sequence that is homologous to the fragment to which it is to be joined.

5. The method according to claim 1, wherein at least one of the two or more nucleic acid fragments is prepared using the Polymerase Chain Reaction (PCR).

6. The method according to claim 1, wherein the first virus is from the family Poxviridae.

7. The method according to claim 6, wherein the first virus is from the genus orthopoxvirus.

8. The method according to claim 7, wherein the first virus is from the species vaccinia virus.

9. The method according to claim 6, wherein the first virus is from the genus leporipoxvirus.

10. The method according to claim 1, wherein the second virus is from the family Poxviridae.

11. The method according to claim 10, wherein the second virus is from the genus leporipoxviruses.

12. The method according to claim 11 wherein the second virus is from the species Shope fibroma virus.

13. The method according to claim 1, wherein the first recombinant virus is recovered by plating the host cells, or an extract therefrom, on a cell line that does not support the replication of the second virus.

14. A method according to claim 13 wherein the cell line is selected from African green monkey cells or HeLa cells.

15. A method according to claim 1 wherein the recombinant virus is recovered at a concentration of greater than 10² PFU/μg.

16. A method according to claim 15 wherein the recombinant virus is recovered at a concentration of greater than 10⁶ PFU/μg.

17. A method according to claim 1 wherein the nucleic acid fragments are at least 50 bp in length.

18. A method according to claim 1 wherein the nucleic acid fragments are from about 500 bp to 20,000 bp in length.

19. A method according to claim 1 for producing a first recombinant virus comprising a heterologous nucleic acid sequence encoding a foreign gene of interest comprising:
(a) providing a host cell that is infected with a second virus;
(b) introducing (i) two or more nucleic acid fragments from the first virus into the host cell, wherein said two or more nucleic acid fragments have ends that are capable of being joined and (ii) a heterologous nucleic acid sequence encoding a foreign gene of interest;
(c) incubating the host cell under conditions to allow the nucleic acid fragments to recombine and form a recombinant virus comprising the heterologous nucleic acid sequence; and
(d) recovering the recombinant virus.

20. A method according to claim 1 for producing a first recombinant virus having a deletion in a non-essential region comprising:
(a) providing a host cell that is infected with a second virus;
(b) introducing two or more nucleic acid fragments from the first virus into the host cell, wherein said two or more nucleic acid fragments have ends that are capable of being joined, wherein said fragments do not comprise a non-essential region of the virus;
(c) incubating the host cell under conditions to allow the nucleic acid fragments to recombine and form a recombinant virus having a deletion in a non-essential region; and
(d) recovering the recombinant virus.

21. A method according to claim 1 for producing a first recombinant virus comprising:
(a) extracting nucleic acids from a first virus;
(b) preparing fragments of the nucleic acids and separating the fragments into different containers wherein each container will not contain a sufficient number of fragments to prepare an active first virus;
(c) optionally, storing the containers;
(d) providing a host cell that is infected with a second virus;
(e) introducing two or more nucleic acid fragments from at least two different containers into the host cell, wherein said two or more nucleic acid fragments have ends that are capable of being joined;
(f) incubating the host cell under conditions to allow the nucleic acid fragments to recombine and form a recombinant virus; and
(g) recovering the recombinant virus.

22. A kit for carrying out the methods of claim 1 comprising a host cell and a second virus suitable for packaging a first virus into infectious virions.

23. The kit according to claim 22 further comprising a DNA sequence comprising the first viral genome, restriction enzymes to cut the first viral genome at unique site(s) and/or reagents to perform the PCR reaction.

24. The kit according to claim 23 further comprising a cell line that does not support the replication of the second virus.
